(19) United States
(12) Patent Application Publication
(10) Pub. No.: US 2012/0101590 A1
(43) Pub. Date: Apr. 26, 2012

(54) CERAMIC BALL SOCKET INSERT HAVING INVERSE-CONICAL GUIDE PINS

(76) Inventors: Roman Preuss, Kirchheim unter Teck (DE); Thomas Pandorf, Wernau (DE); Yvonne Bläske, Backnang (DE); Thamas Bögel, Wernau (DE)

(21) Appl. No.: 13/147,616

(22) PCT Filed: Feb. 4, 2010
(86) PCT No.: PCT/EP2010/051371

§ 371 Date: Jan. 12, 2012

(30) Foreign Application Priority Data
Feb. 11, 2009 (DE) 10 2009 000 771.7

(51) Int. Cl. A61F 2/36

(52) U.S. Cl. 623/23.11

(57) ABSTRACT

A hip joint prosthesis having a socket insert and a hip socket, a conical guide pin being situated on the pole of the socket insert and a guide bore being situated at the base of the hip socket and the guide pin being located in the guide bore in the installed state of the socket insert in the hip socket,

The invention describes a hip joint prosthesis having a socket insert and a hip socket, a conical guide pin being situated on the pole of the socket insert and a guide bore being situated at the base of the hip socket, and the guide pin being located in the guide bore in the installed state of the socket insert in the hip socket.

A number of prosthetic systems for replacement of the natural hip joint currently exist on the market. These prosthetic systems are generally composed of a shank 1 which is coupled to a ball head 2, and a hip socket 4 which is coupled to a socket insert 3 (see FIG. 1). The shank 1 and the hip socket 4 are joined to the body by ingrowth into the femur and the pelvic bone, respectively, and are supports for the ball head 2 and socket insert 3, respectively. The ball head 2 is rotatably supported in the spherical cap of the socket insert 3 with one degree of freedom. The shank 1 and the ball head 2 are generally coupled by conical clamping. This usually applies to the socket insert 3 and the hip socket 4 as well, which as a rule are also coupled by conical clamping. FIG. 1 shows a hip prosthesis comprising a shank 1, ball head 2, hip socket 3, and socket insert 4.

During the insertion process, in particular of thin-walled metal sockets into the pelvic bone, deformation of the metal sockets may occur in the region of the clamping cone, thus making the correct, functionally proper insertion of the conically clamped socket insert more difficult. In the extreme case, the socket insert and hip socket become jammed in a tilted position of the socket insert in the hip socket. The tilting of the socket insert changes the load conditions, and results in concentrated loads which may significantly reduce the durability of the clamping connection as well as the service life of the socket insert itself.

In addition, in particular for minimally invasive surgical procedures, visibility in the surgical area is generally inadequate, for example, to correctly insert the socket insert into the hip socket with visual control. The conical clamping of the socket insert in the hip socket is self-centering during the insertion. However, this guiding of the socket insert during insertion into the hip socket is effective only at small initial tilting angles. If greater initial tilting occurs due to limited visibility for the surgeon, the self-centering fails, and tilted clamping with the above-described consequences occurs.

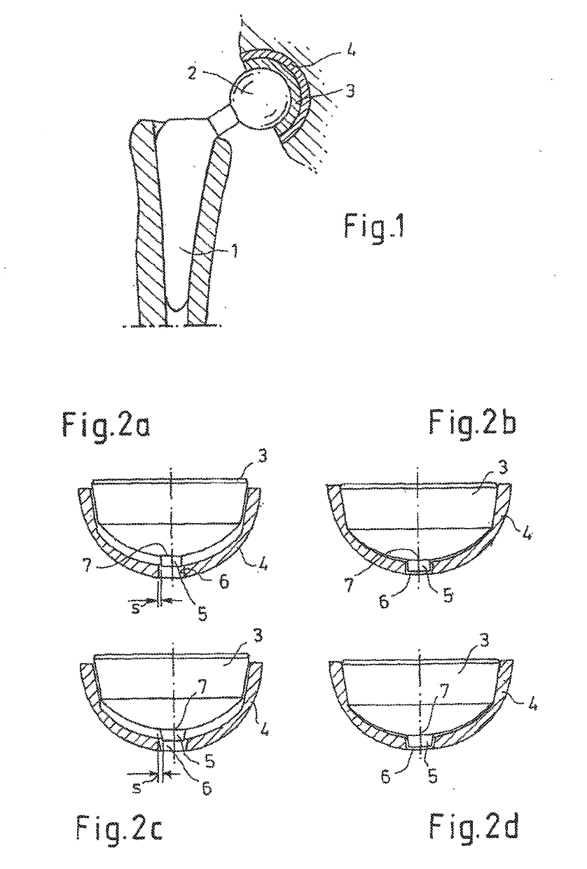

For this reason, heretofore additional guiding of the socket insert has been performed during the insertion motion for various socket inserts. For this purpose, a cylindrical (see FIGS. 2a, 2b) or conical (see FIGS. 2c, 2d) guide pin is provided on the pole of the particular socket insert 3. When the socket insert 3 is inserted into a correspondingly shaped guide bore 6, the guide pin is introduced at the base of the hip socket 4, thus preventing tilting of the socket insert 3. FIGS. 2a, 2b, 2c, 2d show a socket insert 3 having a cylindrical (upper) or conical (lower) guide pin 5 at the rear pole. The gap width s (see FIGS. 2a, 2c) determines the degree of accuracy with which the socket insert 3 is guided during insertion into the hip socket 4.

Due to the small installation space for the guide pin 5 and the guide bore 6, the guide length of the guide pin 5 is generally extremely small, in particular at the moment that the socket insert is inserted; however, when the length of the guide pin inside the guide bore is still small, the need for guiding for proper insertion of the socket insert is greatest. The guide length increases with progressive insertion of the socket insert into the hip socket, with increasing improvement of the guiding effect. However, the guiding effect of the conical clamping connection between the socket insert and the hip socket likewise increases, which progressively reduces the need for guiding by the guide pin. Thus, there is an inverse relationship between the need for guiding and the guiding accuracy of the guide pin.

An appropriately large guide gap s must be achieved in order to avoid tilting of the guiding. In addition, the diameter tolerances of the guide pin and the guide bore dictate a necessary minimum size of the guide gap. However, as the size of the guide gap increases, the guiding accuracy between the guide pin and the guide bore decreases, and the risk of tilting of the socket insert in the hip socket increases.

The object of the invention is to refine a hip joint prosthesis, a socket insert, and a hip socket according to the preambles of claims 1, 6, and 7, respectively, in such a way that proper insertion of the socket insert into the hip socket with high guidance accuracy is made possible, even under difficult conditions.

This object is achieved according to the invention by the features of claims 1, 6, and 7.

According to the invention, the guide pin has an inverse conically tapered design, the diameter of the guide pin at the end facing the pole being smaller than at the end of the guide pin facing away from the pole.

As the result of providing a guide pin on the pole of the socket insert which has an inverse conical taper and which is inserted into a cylindrical or likewise inverse conical guide bore in the base of the hip socket, the theoretical guide length of the system becomes zero, and a departure is made from the principle of the design of classical guiding. However, guiding, and therefore support, of the insertion of the socket insert in order to avoid the tilted position still occurs. In addition, extremely small guide gaps and therefore high guiding accuracy are achievable. In particular, the small guide gap and therefore the high guiding accuracy are achieved when the socket insert is first inserted into the hip socket.

As described, one embodiment according to the invention is characterized in that the guide bore has an inverse conically tapered design, the diameter of the guide bore at the end facing the interior, i.e., the base of the hip socket, being smaller than at the end of the guide bore facing away from the interior.

Another embodiment according to the invention is characterized in that the guide bore has a cylindrical design.

The guide bore may also preferably be composed of two sections, the guide bore being cylindrical in the first section and having an inverse conical tapered design in the second section.

The first section is advantageously situated at the end of the guide bore facing the interior of the hip socket.

A socket insert according to the invention for a hip joint prosthesis, having a conical guide pin situated at the pole for insertion into a corresponding guide bore in a hip socket, is characterized in that the guide pin has an inverse conically tapered design, the diameter of the guide pin at the end facing the pole being smaller than at the end of the guide pin facing away from the pole.

A hip socket according to the invention for a hip joint prosthesis, having a conical guide bore situated at the base of the hip socket for accommodating a guide pin of a socket insert, is characterized in that the guide bore has an inverse conically tapered design, the diameter of the guide bore at the end facing the interior of the hip socket being smaller than at the end of the guide bore facing away from the interior.

As a result of the designs according to the invention of the socket insert for a hip joint prosthesis which is provided with a guide pin having an inverse conically tapered design, tilted insertion of the socket insert into the hip socket is avoided due to the fact that guiding for the insertion motion of the socket insert starts at the beginning of the insertion process.

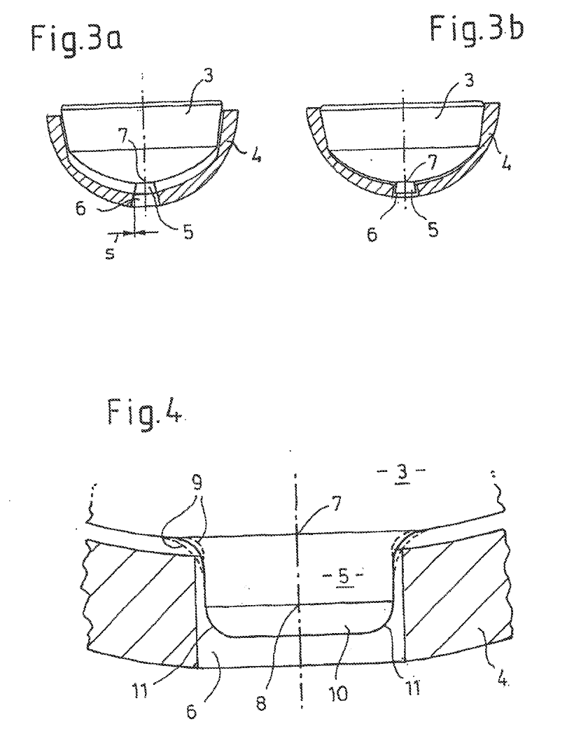

For a cylindrical guide bore, the small guide gap remains constant over the entire length of the guiding, whereas for an inverse conical guide bore the guide gap increases with increasing insertion depth of the socket insert. However, the resulting decrease in the guiding accuracy also corresponds to the likewise decreasing need for guiding, since the conical clamping increasingly achieves the guiding effect. FIGS. 3a, 3b show a socket insert 3 having an inverse conical pin 5 at the rear pole 7. The very small gap width s results in a high guiding effect with a low risk of tilting.

Another positive effect of the inverse conical shape of the pin 5 results when rounded radii are provided on the component. This is necessary when brittle materials are used, for example when the socket insert 3 is made of a ceramic material. In such a case, the edges 9 of the pin 5 must be rounded in order to reduce notch stresses and edge chipping.

Tolerance analyses of rounded radii on cylindrical or conical pins with regard to the maximum and minimum allowable rounded radius show that collisions sometimes occur with the borehole in the socket pole. These may be avoided only by limiting tolerances, or by reducing the pin diameter (see FIG. 4). Tolerance limitations generally increase the manufacturing costs. Reducing the pin diameter increases the gap width s and reduces the guiding effect of the pin 5 when the socket insert is inserted. FIG. 4 shows a socket insert having a cylindrical pin 5 and rounded edges 9. Tolerance analysis with regard to the maximum and minimum edge radius (dashed lines) shows a collision with the hip socket in the region of the through borehole having the largest radius.

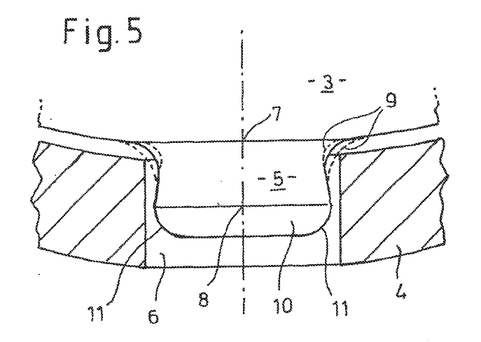

For an inverse conical shape of the pin 5, the tapering of the pin results in an enlarged installation space at the end of the pin on the insertion side. For the same values, tolerance analyses of the edge rounding thus result in a greater distance from the through borehole or guide bore 6 in the hip socket 4. The guiding effect of the pin is maintained without the occurrence of undesired collisions between the components (see FIG. 5). FIG. 5 shows a socket insert having an inverse conical pin and rounded edges 9. Tolerance analysis with regard to the maximum and minimum edge radius (dashed lines) shows no collision with the hip socket. The “pin” and the “guide pin” are two separate terms which, however, denote the same subject matter.

The end region 10 of the guide pin 5 or pin facing away from the pole 7 is rounded; i.e., the inverse conically tapered guide pin 5 has a rounded end region 10 (see FIGS. 4 and 5). This simplifies, among other things, the insertion into the guide bore 6.

1-9. (canceled)

10. A hip joint prosthesis having a socket insert and a hip socket, a conical guide pin being situated on the pole of the socket insert and a guide bore being situated at the base of the hip socket, and the guide pin being located in the guide bore in the installed state of the socket insert in the hip socket, wherein the guide pin has an inverse conically tapered design, the diameter of the guide pin at the end facing the pole being smaller than at the end of the guide pin facing away from the pole.

11. A hip joint prosthesis according to claim 10, wherein the guide bore has an inverse conically tapered design such that the diameter of the guide bore at the end facing the interior, is smaller than at the end of the guide bore facing away from the interior.

12. A hip joint prosthesis according to claim 10, wherein the guide bore has a cylindrical design.

13. A hip joint prosthesis according to claim 10, wherein the guide bore is composed of two sections wherein a first section of the guide bore is cylindrical and wherein a second section of the guide bore has an inverse conical tapered design.

14. A hip joint prosthesis according to claim 11, wherein the guide bore is composed of two sections wherein a first section of the guide bore is cylindrical and wherein a second section of the guide bore has an inverse conical tapered design.

15. A hip joint prosthesis according to claim 13, wherein the first section is situated at the end of the guide bore facing the interior of the hip socket.

16. A hip joint prosthesis according to claim 14, wherein the first section is situated at the end of the guide bore facing the interior of the hip socket.

17. A socket insert for a hip joint prosthesis, having a conical guide pin situated at the pole for insertion into a corresponding guide bore in a hip socket, wherein the guide pin has an inverse conically tapered design, the diameter of the guide pin at the end facing the pole being smaller than at the end of the guide pin facing away from the pole.

18. A hip socket for a hip joint prosthesis, having a conical guide bore situated at the base of the hip socket for accommodating a guide pin of a socket insert, wherein the guide bore has an inverse conically tapered design, the diameter of the guide bore at the end facing the interior of the hip socket being smaller than at the end of the guide bore facing away from the interior.

19. A hip joint prosthesis according to claim 10, wherein the socket insert is made of a ceramic, and the guide pin has rounded edges.

20. A socket insert according to claim 17, wherein the socket insert is made of a ceramic, and the guide pin has rounded edges.

21. A hip joint prosthesis according to claim 10, wherein the guide pin has a rounded end region.

22. A socket insert according to claim 17, wherein the guide pin has a rounded end region.

23. A hip joint prosthesis according to claim 11, wherein the socket insert is made of a ceramic, and the guide. pin has rounded edges.

24. A hip joint prosthesis according to claim 12, wherein the socket insert is made of a ceramic, and the guide pin has rounded edges.

25. A hip joint prosthesis according to claim 13, wherein the socket insert is made of a ceramic, and the guide pin has rounded edges.

26. A hip joint prosthesis according to claim 14, wherein the socket insert is made of a ceramic, and the guide pin has rounded edges.

27. (ne A hip joint prosthesis according to claim 15, wherein the socket insert is made of a ceramic, and the guide pin has rounded edges.

28. A hip joint prosthesis according to claim 16, wherein the socket insert is made of a ceramic, and the guide pin has rounded edges.
